(19) United States
(12) Patent Application Publication
(10) Pub. No.: US 2023/0301567 A1
(43) Pub. Date: Sep. 28, 2023

(54) CATHETER SYSTEM INSERT

(71) Applicant: Becton, Dickinson and Company, Franklin Lakes, NJ (US)

(72) Inventors: Xiwei Chen, Shanghai (CN); Bo Yan, Shanghai (CN)

(21) Appl. No.: 18/033,722

(22) PCT Filed: Oct. 19, 2021
(86) PCT No.: PCT/US2021/055599

§ 371 Date: Apr. 25, 2023

(30) Foreign Application Priority Data
Oct. 29, 2020 (CN) 202011175769.9

(51) Int. Cl. A61B 5/15

(52) U.S. Cl. CPC A61B 5/15003; A61B 5/150992; A61M 25/0067

(57) ABSTRACT

An insert for a catheter system may include a distal end, a proximal end, and a middle portion disposed between the distal end and the proximal end. The distal end of the insert may include a male port, and the proximal end of the insert may include a female port opposite the male port. The insert may be configured to extend through an adapter and/or a needleless connector of a catheter system to reduce exposure of the adapter and/or the needleless connector to blood, which may flow through a lumen of the insert. The lumen of the insert may extend through the male port, the middle portion, and the female port.

### BACKGROUND

Intravenous catheters are commonly used for a variety of infusion therapies. For example, intravenous catheters may be used for infusing fluids, such as normal saline solution, various medicaments, and total parenteral nutrition, into a patient. Intravenous catheters may also be used for withdrawing blood from the patient.

Common types of intravenous catheter are peripheral IV catheters (“PIVCs”), peripherally inserted central catheters (“PICCs”), and midline catheters. Intravenous catheters may include “over-the needle” catheters, which may be mounted over a needle having a sharp distal tip. The sharp distal tip may be used to pierce skin and the vasculature of the patient. Insertion of the intravenous catheter into the vasculature may follow the piercing of the vasculature by the needle. The needle and the intravenous catheter are generally inserted at a shallow angle through the skin into the vasculature of the patient with a bevel of the needle facing up and away from the skin of the patient.

In order to verify proper placement of the introducer needle and/or the intravenous catheter in the vasculature, a user generally confirms that there is flashback of blood, which may be visible to the user. In some instances, the introducer needle may include a notch disposed towards a distal end of the introducer needle, and in response to the distal tip of the introducer needle being positioned within the vasculature, blood may flow proximally through a needle lumen, exit the needle lumen through the notch, and then travel proximally between an outer surface of the introducer needle and an inner surface of the intravenous catheter.

Accordingly, where the intravenous catheter is at least partially transparent, the user may visualize a small amount of blood “flashback” and thereby confirm placement of the intravenous catheter within the vasculature. Presence of a vasculature entrance indicator, such as flashback, may facilitate successful placement of intravenous catheters. Once placement of the introducer needle within the vasculature has been confirmed, the user may temporarily occlude flow in the vasculature and withdraw the introducer needle, leaving the intravenous catheter in place for future blood withdrawal and/or fluid infusion.

During blood withdrawal, blood may travel through an adapter and/or needleless connector in fluid communication with the intravenous catheter via an extension tube. After a blood sample is collected, to prevent contamination by the blood, a user typically flushes the adapter with saline and replaces the needleless connector. Flushing the adapter and replacing the needleless connector add additional steps of operation for the user and may increase a risk of blood exposure for the user. Moreover, there is a risk of hemolysis when blood travels through a non-smooth, uneven pathway of the needleless connector.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure generally relates to an insert and related systems and methods. In some embodiments, the insert for a catheter system may include a distal end, a proximal end, and a middle portion disposed between the distal end and the proximal end. In some embodiments, the distal end of the insert may include a male port, and the proximal end of the insert may include a female port opposite the male port. In some embodiments, the insert may be configured to extend through an adapter and/or a needleless connector of the catheter system to reduce exposure of the adapter and/or the needleless connector to blood, which may flow through a lumen of the insert. In some embodiments, the lumen of the insert may extend through the male port, the middle portion, and the female port.

In some embodiments, the middle portion may include an elongated tubular portion and a tapered portion or a stepped portion. In some embodiments, the tapered portion or the stepped portion may be proximal and proximate to the elongated tubular portion. In some embodiments, an outer diameter of the elongated tubular portion may be uniform. In some embodiments, an outer diameter of the tapered portion may increase in a proximal direction.

In some embodiments, the insert may include a septum, which may be disposed within the female port. In some embodiments, the septum may seal the female port. In some embodiments, the septum may include a split septum. In some embodiments, the insert may be integrally formed or monolithically formed as a single unit. In some embodiments, the elongated tubular portion may have a greater length than the tapered or stepped portion.

In some embodiments, the catheter system may include a catheter assembly, which may include one or more of the following: a catheter adapter; a catheter extending distally from the catheter adapter; an extension tube; an adapter coupled to a proximal end of the extension tube; and a needleless connector coupled to the adapter. In some embodiments, the extension tube may include the proximal end and a distal end, which may be coupled to the catheter adapter. In some embodiments, the insert may be inserted through the needleless connector and the adapter such that the male port is disposed within the extension tubing and/or the tapered or stepped portion is proximal to the needleless connector. In some embodiments, in response to the insert being inserted through the needleless connector and the adapter of the catheter system, blood may be configured to flow through the insert and may not contact the needleless connector or the adapter.

In some embodiments, the catheter system may include a needle assembly and/or a blood collection tube holder coupled to the needle assembly. In some embodiments, the needle assembly may include a body, which may include a distal end, a proximal end, and a lumen extending through the distal end of the body and the proximal end of the body. In some embodiments, the distal end of the body may include a male port. In some embodiments, the needle assembly may include a needle extending from the proximal end of the body and configured to receive an evacuated blood collection tube.

In some embodiments, a method of blood sampling may include inserting the insert through the needleless connector and the adapter of the catheter system. In some embodiments, the insert may be inserted through the needleless connector and the adapter such that the male port is disposed within the extension tubing and at least a portion of the tapered portion or the stepped portion is proximal to the needleless connector.

In some embodiments, the method may include inserting the needle assembly into the female port of the insert. In some embodiments, after inserting the insert through the needleless connector and the adapter and after inserting the needle assembly into the female port of the insert, the catheter may be inserted into a vein of a patient. In some embodiments, the catheter assembly may include an introducer needle, which may extend through the catheter and may be used to insert the catheter into the vein. In some embodiments, after the catheter is inserted into the vein, the introducer needle may be removed from the catheter system and patient.

In some embodiments, after inserting the catheter into the vein of the patient, the needle may be inserted into the evacuated blood collection tube and blood may be collected in the evacuated blood collection tube. In some embodiments, after blood is collected in the evacuated blood collection tube, the needle assembly may be removed from the female port of the insert and a syringe may be coupled to the female port.

In some embodiments, the female port may be threaded. In some embodiments, coupling the syringe to the female port may include threading the syringe to the female port. In some embodiments, the catheter and the catheter assembly may be flushed by activating or depressing the syringe to release fluid in the syringe into the catheter assembly. In some embodiments, after the catheter is flushed, the insert may be removed from the catheter system during an indwelling period of the catheter in the vein of the patient or while the catheter is left within the vein of the patient.

It is to be understood that both the foregoing general description and the following detailed description are explanatory examples and are not restrictive of the invention outlined in the claims. It should be understood that the various embodiments are not limited to what is depicted in the drawings. It should also be understood that the embodiments may be combined, other embodiments may be used, and that structural changes, unless so claimed, may be made without departing from the scope of the various embodiments of the present invention. The following detailed description is therefore not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example insert, according to some embodiments;

FIG. 1B is a cross-sectional view of the insert, according to some embodiments;

FIG. 2 is a partially exploded view of an example catheter system, according to some embodiments;

FIG. 3A is an upper perspective view of the catheter system, according to some embodiments;

FIG. 3B is a cross-sectional view of the catheter system, according to some embodiments;

FIG. 4 is an upper perspective view of the catheter system, illustrating an example blood collection device coupled to the catheter system, according to some embodiments;

FIG. 5 is an upper perspective view of the catheter system, illustrating an example syringe coupled to the catheter system, according to some embodiments; and

FIG. 6 is an upper perspective view of the catheter system, illustrating the catheter system after removal of the insert, according to some embodiments.

### DESCRIPTION OF EMBODIMENTS

All examples and conditional language recited herein are intended to aid the reader in understanding the invention and concepts contributed by the inventor to furthering the art; the examples and language are thus to be construed as being not limited to such specifically recited examples and conditions. Although embodiments of the present invention have been described in detail, it should be understood that various changes, substitutions, and alterations could be made hereto without departing from the spirit and scope of the invention.

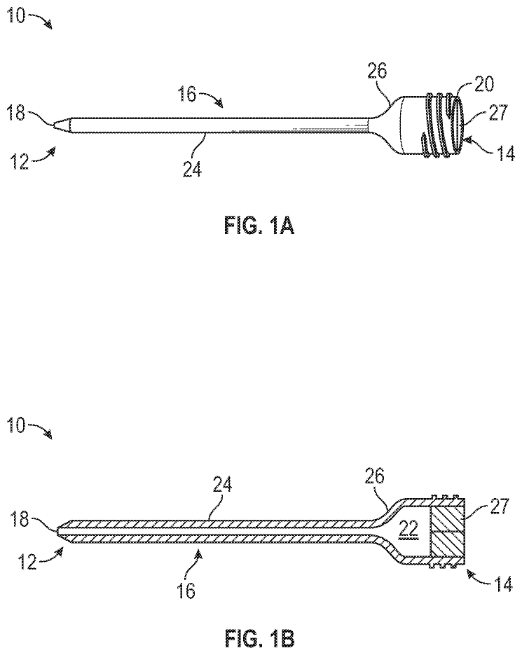

Referring now to FIGS. 1A-1B, in some embodiments, an insert 10 for a catheter system may include a distal end 12, a proximal end 14, and a middle portion 16 disposed between the distal end 12 and the proximal end 14. In some embodiments, the distal end 12 of the insert 10 may include a male port 18, and the proximal end 14 of the insert 10 may include a female port 20 opposite the male port 18. In some embodiments, the insert 10 may be configured to extend through an adapter and/or a needleless connector of the catheter system to reduce exposure of the adapter and/or the needleless connector to blood, which may flow through a lumen 22 of the insert 10. In some embodiments, the lumen 22 of the insert 10 may extend through the male port 18, the middle portion 16, and the female port 20. In some embodiments, the female port 20 may be threaded, which may facilitate coupling to a flush device.

In some embodiments, the middle portion 16 may include an elongated tubular portion 24 and an enlarged diameter portion 26, which may include a tapered portion or a stepped portion. In some embodiments, the enlarged diameter portion 26 may be proximal and proximate to the elongated tubular portion 24. In some embodiments, an outer diameter of the elongated tubular portion 24 may be uniform. In some embodiments, the elongated tubular portion 24 and a portion of the lumen 22 extending there through may be straight, which reduce a risk of hemolysis in the insert 10 compared to the needleless connector. In some embodiments, when the enlarged diameter portion 26 includes the tapered portion, an outer diameter of the tapered portion may increase in a proximal direction.

In some embodiments, the insert 10 may include a septum 27, which may be disposed within the female port 20. In some embodiments, the septum 27 may seal the female port 20. In some embodiments, the septum 27 may include a split septum, which may include a split membrane. In some embodiments, the insert 10 may be integrally formed or monolithically formed as a single unit. In some embodiments, the elongated tubular portion 24 may have a greater length than the enlarged diameter portion 26.

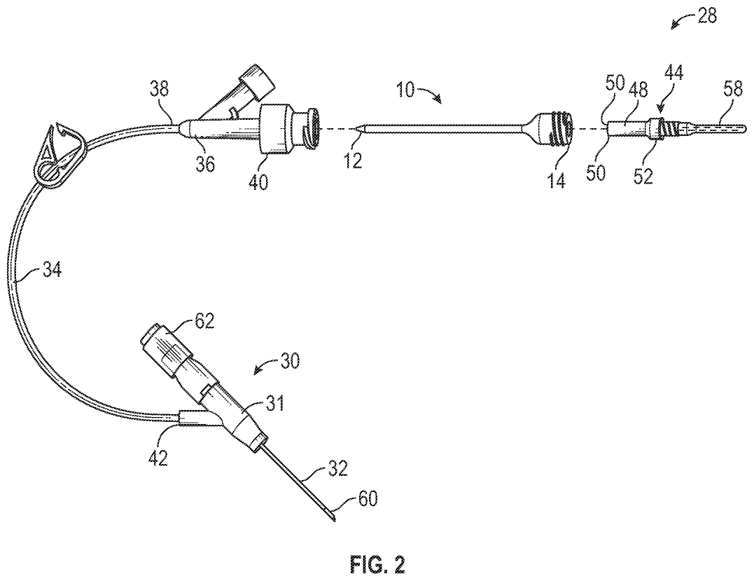

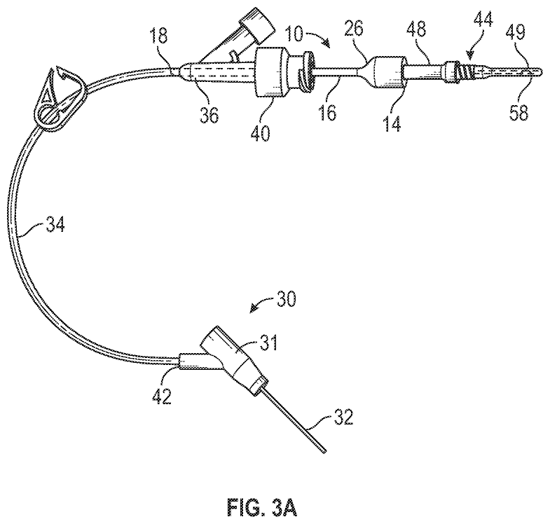

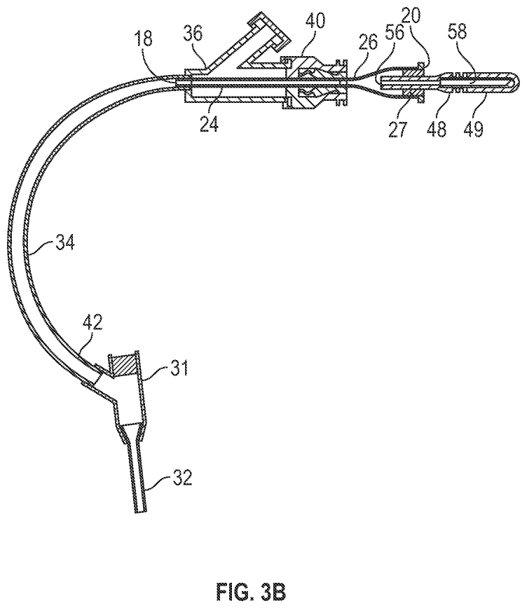

Referring now FIGS. 2-3B, in some embodiments, the catheter system 28 may include a catheter assembly 30, which may include one or more of the following: a catheter adapter 31; a catheter 32 extending distally from the catheter adapter 31; an extension tube 34; an adapter 36 coupled to a proximal end 38 of the extension tube 34; and a needleless connector 40. In some embodiments, the needleless connector 40 may be coupled to the adapter 36.

In some embodiments, the extension tube 34 may include the proximal end 38 and a distal end 42, which may be coupled to the catheter adapter 31. In some embodiments, the extension tube 34 may be coupled to or integrated with a side port of the catheter adapter 31 or another portion of a particular catheter adapter. In some embodiments, the needleless connector 40 may include a BD Q-SYTE™ connector or another suitable needleless connector. In some embodiments, the adapter 36 may include a Y-adapter, a straight adapter, or another suitable adapter.

In some embodiments, the insert 10 may be inserted through the needleless connector 40 and/or the adapter 36 such that the male port 18 is disposed within the extension tube 34. In some embodiments, the male port 18 may be disposed within the extension tube 34 within the adapter 36 and may not extend distal to the adapter 36. In some embodiments, the insert 10 may be pushed or inserted into the adapter 36 until an internal shape of the adapter 36 and/or the extension tubing 34 may prevent the insert 10 from moving further in a distal direction. In some embodiments, the male port 18 may be disposed within the adapter 36 proximate the extension tube 34. In these and other embodiments, blood may be prevented from contacting the adapter 36 and/or the needleless connector 40. In some embodiments, an outer diameter of the insert 10 may be equal to or approximately equal to an inner diameter of the extension tube 34 and/or the adapter 36 such that a seal is formed between the insert 10 and the extension tube 34 and/or the adapter 36, preventing blood from entering or contacting the adapter 36.

In some embodiments, the insert 10 may be inserted through the needleless connector 40 and/or the adapter 36 such that enlarged diameter portion 26 is proximal to and/or proximate the needleless connector 40. In some embodiments, in response to the insert 10 being inserted through the needleless connector 40 and the adapter 36 of the catheter system 28, blood may be configured to flow through the insert 10 and may not contact the needleless connector 40 and/or the adapter 36.

In some embodiments, the catheter system 28 may include a needle assembly 44 and/or a blood collection tube holder (not illustrated) coupled to the needle assembly 44. In some embodiments, the needle assembly 44 may include a body 48, which may include a distal end 50, a proximal end 52, and a lumen 54 extending through the distal end 50 of the body 48 and the proximal end 52 of the body 48. In some embodiments, the distal end 50 of the body 48 may include a male port 56. In some embodiments, the needle assembly 44 may include a needle 58 extending from the proximal end of the body 48 and configured to receive an evacuated blood collection tube.

In some embodiments, an elastomeric sheath 49 may be coupled to the body 48 of the needle assembly 44. In some embodiments, a proximal end of the needle 58 may be enveloped within the elastomeric sheath 49. In some embodiments, the elastomeric sheath 49 may include an open distal end and a closed proximal end. In some embodiments, in response to the evacuated blood collection tube pushing the elastomeric sheath 49 distally, the needle 58 may pierce the elastomeric sheath 49, and the needle 58 may insert into a cavity of the evacuated blood collection tube.

In some embodiments, a method of blood sampling may include inserting the insert 10 through the needleless connector 40 and/or the adapter 36 of the catheter system 28. In some embodiments, the method may include coupling the needle assembly 44 to the female port 20 of the insert 10. In further detail, in some embodiments, the method may include inserting the needle assembly 44 into the female port 20 of the insert 10. For example, the distal end 50 of the body 48 may be inserted into the female port 20. In some embodiments, after inserting the insert 10 through the needleless connector 40 and/or the adapter 36 and after inserting the needle assembly 44 into the female port 20 of the insert 10, the catheter 32 may be inserted into a vein of a patient.

As illustrated in FIG. 2, in some embodiments, the catheter system 28 may include an introducer needle 60, which may extend through the catheter 32 and may be used to insert the catheter 32 into the vein. In some embodiments, a proximal end of the introducer needle 60 may be secured within a needle hub 62. In some embodiments, after the catheter 32 is inserted into the vein, the introducer needle 60 may be removed from the catheter system 28 and the patient.

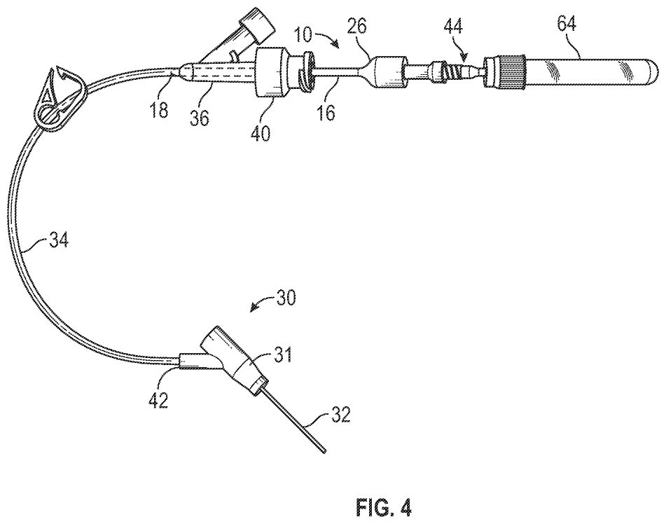

Referring now to FIG. 4, in some embodiments, after inserting the catheter 32 into the vein of the patient, the needle 58 may be inserted into the evacuated blood collection tube 64 and blood may be collected in the evacuated blood collection tube 64. When the patient's blood is collected via the insert 10 in accordance with some embodiments, both the adapter 36 and the needleless connector 40 may be bypassed and thereby avoid contamination.

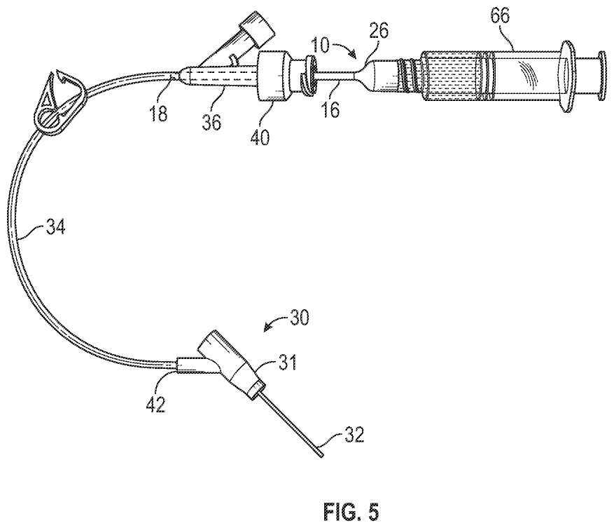

Referring now to FIG. 5, in some embodiments, after blood is collected in the evacuated blood collection tube 64, the needle assembly 44 may be removed from the female port 20 of the insert 10 and a syringe 66 or another flush device may be coupled to the female port 20. In some embodiments, coupling the syringe 66 to the female port 20 may include threading the syringe 66 to the female port 20. In some embodiments, the syringe 66 may be coupled to the female port 20 via another suitable mechanism. In some embodiments, the catheter 32 and the catheter assembly 30 may be flushed by activating or depressing the syringe 66 to release fluid in the syringe 66 into the catheter assembly 30.

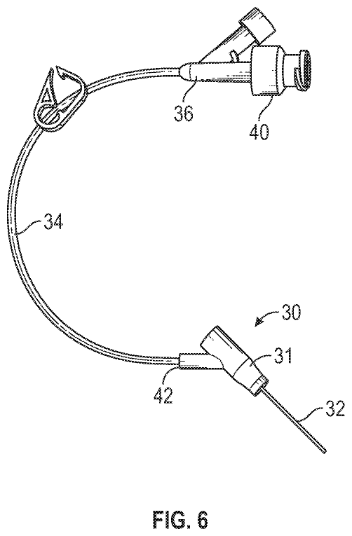

Referring now to FIG. 6, in some embodiments, after the catheter 32 is flushed, the insert 10 may be removed from the catheter system 28 during an indwelling period of the catheter 32 in the vein of the patient or while the catheter 32 is left within the vein of the patient.

The present invention may be embodied in other specific forms without departing from its structures, methods, or other essential characteristics as broadly described herein and claimed hereinafter. The described embodiments are to be considered in all respects only as illustrative and not restrictive. The scope of the invention is therefore indicated by the appended claims rather than by the foregoing description. All changes that come within the meaning and range of equivalency of the claims are to be embraced within their scope.

We claim:

1. An insert for a catheter system, the insert comprising:
a distal end, comprising a male port;
a proximal end, comprising a female port opposite the male port;
a middle portion disposed between the distal end and the proximal end, wherein the middle portion comprises an elongated tubular portion and a tapered or stepped portion proximal and proximate to the elongated tubular portion, wherein an outer diameter of the elongated tubular portion is uniform, wherein an outer diameter of the tapered or stepped portion increases in a proximal direction; and
a lumen extending through the male port, the middle portion, and the female port.

2. The insert of claim 1, further comprising a septum disposed within the female port.

3. The insert of claim 2, wherein the septum comprises a split septum.

4. The insert of claim 1, wherein the insert is monolithically formed as a single unit.

5. The insert of claim 1, wherein the elongated tubular portion has a greater length than the tapered or stepped portion.

6. A catheter system, comprising:
a catheter assembly, comprising:
a catheter adapter;
a catheter extending distally from the catheter adapter;
an extension tube, comprising a distal end and a proximal end, wherein the distal end is coupled to the catheter adapter;
an adapter coupled to the proximal end of the extension tube; and
a needleless connector coupled to the adapter; and
an insert, comprising:
a distal end, comprising a male port;
a proximal end, comprising a female port opposite the male port;
a middle portion disposed between the distal end and the proximal end, wherein the middle portion comprises an elongated tubular portion and a tapered or stepped portion proximal and proximate to the elongated tubular portion, wherein an outer diameter of the elongated tubular portion is uniform, wherein an outer diameter of the tapered or stepped portion increases in a proximal direction; and
a lumen extending through the male port, the middle portion, and the female port, wherein the insert is inserted through the needleless connector and the adapter such that the male port is disposed within the extension tube and the tapered or stepped portion is proximal to the needleless connector.

7. The catheter system of claim 6, wherein the insert further comprises a septum disposed within the female port.

8. The catheter system of claim 7, wherein the septum comprises a split septum.

9. The catheter system of claim 6, wherein in response to inserting the insert through the needleless connector and adapter of the catheter system, blood is configured to flow through the insert and does not contact the needleless connector or the adapter.

10. The catheter system of claim 6, further comprising a needle assembly and a blood collection tube holder coupled to the needle assembly, wherein the needle assembly comprises a body, comprising a distal end, a proximal end, and a lumen extending through the distal end of the body and the proximal end of the body, wherein the distal end of the body comprises a male port, wherein the needle assembly further comprises a needle extending from the proximal end of the body and configured to receive an evacuated blood collection tube.

11. The catheter system of claim 6, wherein the insert is monolithically formed as a single unit.

12. The catheter system of claim 6, wherein the elongated tubular portion has a greater length than the tapered or stepped portion.

13. A method of sampling blood, comprising:
inserting an insert through a needleless connector and adapter of a catheter system, wherein the catheter system comprises:
a catheter adapter;
a catheter extending distally from the catheter adapter;
an extension tube, comprising a distal end and a proximal end, wherein the distal end is coupled to the catheter adapter;
an adapter coupled to the proximal end of the extension tube; and
a needleless connector coupled to the adapter;
wherein the insert comprises:
a distal end, comprising a male port;
a proximal end, comprising a female port opposite the male port;
a middle portion disposed between the distal end and the proximal end, wherein the middle portion comprises an elongated tubular portion and a tapered or stepped portion proximal and proximate to the elongated tubular portion, wherein an outer diameter of the elongated tubular portion is uniform, wherein an outer diameter of the tapered or stepped portion increases in a proximal direction; and
a lumen extending through the male port, the middle portion, and the female port, wherein the insert is inserted through the needleless connector and the adapter such that the male port is disposed within the extension tube and the tapered or stepped portion is proximal to the needleless connector; and
inserting a needle assembly into the female port of the insert, wherein the needle assembly comprises a body, wherein the body comprises a distal end, a proximal end, and a lumen extending through the distal end of the body and the proximal end of the body, wherein the distal end of the body comprises a male port, wherein the needle assembly further comprises a needle extending from the proximal end of the body and configured to receive an evacuated blood collection tube; and
after inserting the insert through the needleless connector and the adapter and after inserting the needle assembly into the female port of the insert, inserting the catheter into a vein of a patient.

14. The method of claim 12, further comprising, after inserting the catheter into the vein of the patient, inserting the needle into the evacuated blood collection tube and collecting blood in the evacuated blood collection tube.

15. The method of claim 13, further comprising, removing the needle assembly from the female port of the insert and coupling a syringe to the female port.

16. The method of claim 14, wherein the female port is threaded, wherein coupling the syringe to the female port comprises threading the syringe to the female port.

17. The method of claim 15, further comprising, flushing the catheter by activating the syringe.

18. The method of claim 17, further comprising, removing the insert from the catheter system during an indwelling period of the catheter in the vein of the patient.

19. The method of claim 12, wherein in response to inserting the insert through the needleless connector and adapter of the catheter system, blood is configured to flow through the insert and does not contact the needleless connector or the adapter.

20. The method of claim 12, wherein the insert further comprises a septum disposed within the female port.
